(19) United States
(12) Patent Application Publication
(10) Pub. No.: US 2011/0230778 A1
(43) Pub. Date: Sep. 22, 2011

(54) Methods and devices for continual respiratory monitoring using adaptive windowing

(76) Inventors: Yungkai Kyle Lai, Aliso Viejo, CA (US); Yongji Fu, Vancouver, WA (US)

(21) Appl. No.: 12/661,521

(22) Filed: Mar. 18, 2010

(51) Int. Cl. A61B 5/08

(52) U.S. Cl. 600/529

(57) ABSTRACT

Methods and devices for continual respiratory monitoring of a human subject using adaptive windowing provide continual estimates of the respiration period of the subject by continually buffering and evaluating samples of a respiratory signal in which the subject's breath sounds are embodied, and dynamically adjust the sampling window length based at least in part on the respiration period. Through this adaptive windowing technique, a sampling window length is maintained that is tailored to the subject's breathing habits, does not unduly inhibit real-time respiratory monitoring, and does not place unnecessary burdens on memory and processing resources of the respiratory monitoring device.

### BACKGROUND OF THE INVENTION

The present invention relates to continual physiological state monitoring and, more particularly, to continual respiratory monitoring of a human subject.

Continual monitoring the physiological state of people who suffer from chronic diseases is an important aspect of chronic disease management. By way of example, continual respiratory monitoring is in widespread use managing respiratory diseases such as asthma and sleep apnea.

One variable commonly monitored in respiratory monitoring applications is respiration period, which is a measured time of a breathing cycle from the start of inspiration to the end of expiration. In these applications, the respiration period may itself be an output, or may be an input used in determining other outputs, such as whether apnea is occurring. To estimate respiration period, a respiratory monitoring device often buffers and evaluates samples of a respiratory signal in which lung sounds of a person being monitored are embodied, wherein all samples are of a predetermined length, i.e. fixed sampling window length.

Selecting a fixed sampling window length for estimating respiration period presents challenges. The window must be long enough to cover at least one full breathing cycle of the person being monitored. Moreover, it may be beneficial for the window to cover multiple breathing cycles to enable the estimate to overcome short-term signal anomalies, such as high noise and irregular breathing patterns. On the other hand, the longer the window is, the less frequently estimates can be made, which inhibits real-time monitoring. Moreover, the window length must comport with memory and processing constraints of the respiratory monitoring device, which can be severe, especially in ambulatory monitoring devices.

Further complicating the selection of a fixed sampling window length is the high degree of variability in human respiration periods. There is no “typical” human respiration period. For some people, an average respiration period may be as short as two seconds, whereas for others an average respiration period may be as long as 15 seconds. In conventional respiratory monitoring devices, this has routinely led to selection of a long window that can accommodate even the longest respiration period. Unfortunately, selecting the window length to accommodate the abnormal case of the extreme “long breather” can unduly inhibit real-time monitoring and impose unnecessary burdens on memory and processing resources of the respiratory monitoring device.

### SUMMARY OF THE INVENTION

The present invention provides methods and devices for continual respiratory monitoring of a human subject using adaptive windowing. The present methods and devices provide continual estimates of the respiration period of the subject by continually buffering and evaluating samples of a respiratory signal in which the subject's breath sounds are embodied, and dynamically adjust the sampling window length based at least in part on the respiration period. Through this adaptive windowing technique, a sampling window length is maintained that is tailored to the subject's breathing habits, does not unduly inhibit real-time respiratory monitoring, and does not place unnecessary burdens on memory and processing resources of the respiratory monitoring device.

In one aspect of the invention, a method for respiratory monitoring of a human subject using adaptive windowing comprises receiving by a respiratory data processing system a respiratory signal; storing in a signal buffer by the processing system a sample of the respiratory signal, wherein the sample has a length equal to a sampling window length; estimating by the processing system a respiration period based at least in part on the sample; and adjusting by the processing system the sampling window length based at least in part on the respiration period.

In some embodiments, the method further comprises repeating the storing and estimating steps at the adjusted sampling window length.

In some embodiments, the adjusting step is conditioned on an outcome of a comparison of the respiration period with a preceding respiration period estimate.

In some embodiments, the adjusting step comprises multiplying the respiration period by a multiplier.

In some embodiments, the multiplier is determined based at least in part on a physical condition of a human subject being monitored.

In some embodiments, the multiplier is determined based at least in part on signal quality.

In some embodiments, the multiplier is determined based at least in part on the respiration period.

In some embodiments, the method further comprises transmitting by the processing system to a respiratory data output interface information generated based at least in part on the respiration period, and displaying on the output interface the information.

In some embodiments, the method further comprises transmitting by the processing system to an applications interface the sampling window length.

In some embodiments, the respiratory monitoring application comprises one of an apnea monitoring or airway patency monitoring application.

In another aspect of the invention, a respiratory monitoring device comprises a respiratory data capture system, a respiratory data acquisition system communicatively coupled with the capture system, a respiratory data processing system communicatively coupled with the acquisition system, a signal buffer communicatively coupled with the processing system, and a respiratory data output interface communicatively coupled with the processing system, wherein the processing system receives a respiratory signal from the capture system via the acquisition system, stores in the signal buffer a sample of the respiratory signal having a length equal to a sampling window length, estimates a respiration period based at least in part on the sample, adjusts the sampling window length based at least in part on the respiration period, and transmits information generated based at least in part on the respiration period to the output interface, whereon the information is displayed.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a respiratory monitoring device in some embodiments of the invention.

FIG. 2 shows a method for respiratory monitoring of a human subject using adaptive windowing by the respiratory monitoring device of FIG. 1 in some embodiments of the invention.

### DETAILED DESCRIPTION OF A PREFERRED EMBDOIMENT

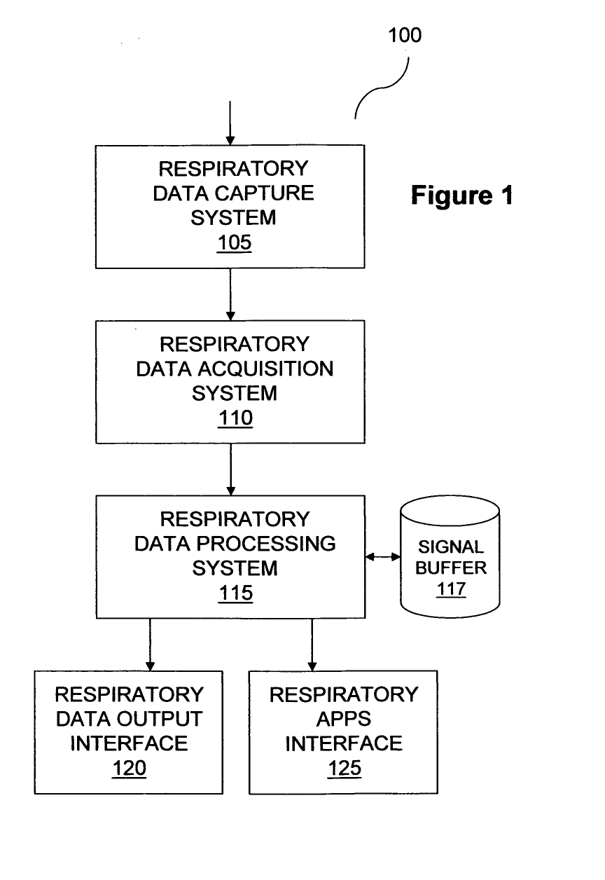

FIG. 1 shows a respiratory monitoring device 100 in some embodiments of the invention. Monitoring device 100 includes a respiratory data capture system 105, a respiratory data acquisition system 110, a respiratory data processing system 115 and a respiratory data output interface 120 communicatively coupled in series. Processing system 115 is also communicatively coupled with a signal buffer 117, and may be communicatively coupled to a respiratory applications interface 125.

Capture system 105 detects lung sounds at a detection point, such as a trachea, chest or back of a person being monitored and transmits a respiratory signal to acquisition system 110 in the form of an electrical signal generated from detected lung sounds. Capture system 105 may include, for example, a sound transducer positioned on the body of a human subject.

Acquisition system 110 amplifies, filters, performs analog/digital (ND) conversion and automatic gain control (AGC) on the respiratory signal received from capture system 105, and transmits the respiratory signal to processing system 115. Amplification, filtering, A/D conversion and AGC may be performed by serially arranged pre-amplifier, band-pass filter, final amplifier, ND conversion and AGC stages, for example.

Processing system 115, under control of a processor executing software instructions, processes the respiratory signal to continually estimate the respiration period of the subject being monitored. To continually estimate the respiration period, processing system 115 continually buffers in signal buffer 117 and evaluates samples of the respiratory signal, wherein the length of each sample is equal to a sampling window length. Processing system 115 under control of the processor transmits information generated based at least in part on the respiratory period to output interface 120. This information may include the respiration period or a respiration rate generated from the respiration period, for example. In addition, processing system 115 may transmit the sampling window length to applications interface 125 for use in other respiratory monitoring applications, such as an apnea or airway patency monitoring application.

In some embodiments, the sampling window is a rectangular window. In these embodiments, data within the window are given equal weight, whereas data outside the window are given no weight, although outside data may be given weight as part of a different sample. Moreover, in some embodiments, the sampling window is non-overlapping, whereas in other embodiments the sampling window is an overlapping, rolling window. Regardless, processing system 115 dynamically adjusts the length of the sampling window based on the respiration period, as will be explained hereinafter in greater detail.

Output interface 120 includes a user interface for displaying information received from processing system 115 generated based at least in part on the respiration period, such as respiration period or respiration rate information. Output interface 120 may also have a data management interface to an internal or external data management system that stores the information and/or a network interface that transmits the information to a remote monitoring device, such as a monitoring device at a clinician facility.

Applications interface 125 is an optional interface that interfaces with one or more respiratory monitoring applications, such as an apnea or airway patency monitoring application, that use sampling window length information received from processing system 115 to facilitate respiratory monitoring.

In some embodiments, capture system 105, acquisition system 110, processing system 115, output interface 120 and applications interface 125 (where present) are part of a portable ambulatory health monitoring device that monitors a person's physiological well-being in real-time as the person performs daily activities. In other embodiments, capture system 105, acquisition system 110, processing system 115, output interface 120 and/or applications interface 125 may be part of separate devices that are remotely coupled via wired or wireless links.

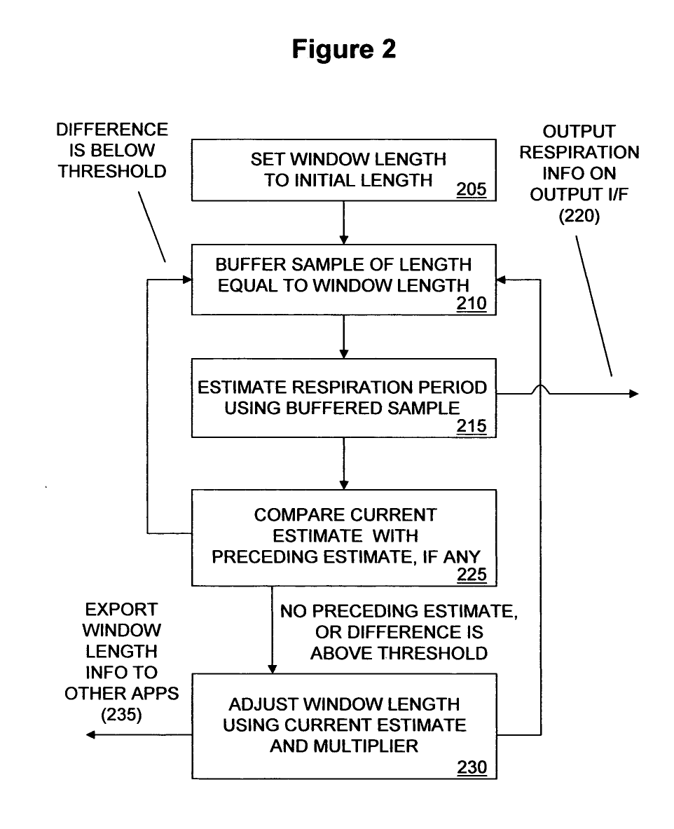

FIG. 2 shows a method for respiratory monitoring of a human subject using adaptive windowing in some embodiments of the invention. In these embodiments, the method is performed by processing system 115 under control of a processor that executes software instructions.

At Step 205, processing system 115 sets the sampling window length to an initial length. In some embodiments, the initial length is selected to ensure that at least one complete respiration period will be captured for a long breather.

At Step 210, processing system 115 stores in signal buffer 117 a sample of the respiratory signal received from capture system 105 via acquisition system 120. The length of the sample is equal to the sampling window length, which at first is the initial length.

At Step 215, processing system 115 estimates the respiration period by evaluating the sample of the respiratory signal stored in signal buffer 117. The respiration period is a measured time of a breathing cycle from the start of inspiration to the end of expiration. In some embodiments, if the sample includes multiple breathing cycles, an average respiration period taken across all cycles is adopted as the estimate. In other embodiments, if the sample includes multiple breathing cycles, the respiration period of the most recent cycle is adopted as the estimate. Moreover, breathing cycles that exhibit poor signal quality or large variance from the norm may be excluded from the estimate.

At Step 220, processing system 115 transmits information generated based on the respiration period estimate to output interface 120, which displays the information on a user screen. By way of example, the transmitted and displayed information may be the respiration period itself, a respiration rate calculated from the respiration period, or a moving average of the respiration period or of the respiration rate calculated from the current respiration period and earlier respiration periods.

At Step 225, processing system 115 compares the current respiration period estimate with the immediately preceding respiration period estimate, if any exists. If there is an immediately preceding estimate (i.e. if the current estimate is not the initial estimate) and the difference between the current and immediately preceding estimates is below a predetermined threshold, the respiratory period is considered stable enough to bypass dynamic adjustment of the sampling window length and the flow returns immediately to Step 210, whereupon a new sample is buffered at the current window length. On the other hand, if the current estimate is the initial estimate, or if the difference between the current and the immediately preceding estimates is above the threshold, the respiratory period is not considered stable enough to bypass dynamic adjustment of the sampling window length and the flow instead advances to Step 230, before returning to Step 210.

At Step 230, processing system 115 adjusts the sampling window length using the current respiration period estimate and a multiplier. By way of example, the multiplier may be statically or dynamically determined based on a physical condition of the person being monitored (e.g. whether the person is a known asthmatic), the quality of the respiratory signal, the length of the current respiration period, and/or the stability of the respiration period. For example, if current signal quality is poor or the respiration period is unstable, the multiplier may be set to a large number such that the sampling window will capture a large number of complete breathing cycles, which can help improve the reliability of the respiration period estimate by taking an average over several cycles. On the other hand, if current signal quality is good and the respiration period is stable, the multiplier may be set to a low number such that sampling window captures a small number of complete breathing cycles, which increases the frequency of respiratory period estimation and reduces burdens on the memory and processing resources of the respiratory monitoring device. Accordingly, the current respiration period estimate and a judiciously selected multiplier result in dynamic tuning of the sampling window to a length that strikes a desired balance between the competing goals of reliable respiration period estimation, on the one hand, and real-time monitoring and memory/processing resource conservation, on the other.

At Step 235, processing system 115 optionally exports the adjusted sampling window length information to applications interface 125, which may use the information in one or more respiratory monitoring applications, such as an apnea or airway patency monitoring application.

It will be appreciated by those of ordinary skill in the art that the invention can be embodied in other specific forms without departing from the spirit or essential character hereof. The present description is therefore considered in all respects to be illustrative and not restrictive. The scope of the invention is indicated by the appended claims, and all changes that come with in the meaning and range of equivalents thereof are intended to be embraced therein.

What is claimed is:

1. A method for respiratory monitoring of a human subject using adaptive windowing, comprising:
receiving by a respiratory data processing system a respiratory signal;
storing in a signal buffer by the processing system a sample of the respiratory signal, wherein the sample has a length equal to a sampling window length;
estimating by the processing system a respiration period based at least in part on the sample; and
adjusting by the processing system the sampling window length based at least in part on the respiration period.

2. The method of claim 1, further comprising repeating the storing and estimating steps at the adjusted sampling window length.

3. The method of claim 1, wherein the adjusting step is conditioned on an outcome of a comparison of the respiration period with a preceding respiration period estimate.

4. The method of claim 1, wherein the adjusting step comprises multiplying the respiration period by a multiplier.

5. The method of claim 4, wherein the multiplier is determined based at least in part on a physical condition of a human subject being monitored.

6. The method of claim 4, wherein the multiplier is determined based at least in part on signal quality.

7. The method of claim 4, wherein the multiplier is determined based at least in part on the respiration period.

8. The method of claim 1, further comprising transmitting by the processing system to a respiratory data output interface information generated based at least in part on the respiration period; and displaying on the output interface the information.

9. The method of claim 1, further comprising transmitting by the processing system to an applications interface the sampling window length, whereupon the sampling window length is used in a respiratory monitoring application.

10. The method of claim 9, wherein the respiratory monitoring application comprises one of an apnea monitoring or airway patency monitoring application.

11. A respiratory monitoring device, comprising:
a respiratory data capture system;
a respiratory data acquisition system communicatively coupled with the capture system;
a respiratory data processing system communicatively coupled with the acquisition system;
a signal buffer communicatively coupled with the processing system; and
a respiratory data output interface communicatively coupled with the processing system, wherein the processing system receives a respiratory signal from the capture system via the acquisition system, buffers in the signal buffer a sample of the respiratory signal having a length equal to a sampling window length, estimates a respiration period based at least in part on the sample, adjusts the sampling window length based at least in part on the respiration period, and transmits information generated based at least in part on the respiration period to the output interface whereon the information is displayed.

12. The device of claim 11, wherein the processing system repeats at the adjusted sampling window length.

13. The device of claim 11, wherein the processing system conditions the adjustment of the sampling window length on an outcome of a comparison of the respiration period with a preceding respiration period estimate.

14. The device of claim 11, wherein the processing system adjusts the sampling window length by multiplying the respiration period by a multiplier.

15. The device of claim 14, wherein the multiplier is determined based at least in part on a physical condition of a human subject being monitored.

16. The device of claim 14, wherein the multiplier is determined based at least in part on signal quality.

17. The device of claim 14, wherein the multiplier is determined based at least in part on the respiration period.

18. The device of claim 14, wherein the processing system transmits to an applications interface the sampling window length, whereupon the sampling window length is used in a respiratory monitoring application.

19. The device of claim 18, wherein the respiratory monitoring application comprises one of an apnea monitoring or airway patency monitoring application.
